(19) United States
(12) Patent Application Publication
(10) Pub. No.: US 2024/0325768 A1
(43) Pub. Date: Oct. 3, 2024

(54) Reducing Electrosensation When Treating a Subject Using a Rotating Alternating Electric Field by Gradually Increasing the Amplitude of the Field

(71) Applicant: Novocure GmbH, Root D4 (CH)

(72) Inventors: Mor BEN-TOV KUPERBERG, Haifa (IL); Yoram WASSERMAN, Haifa (IL)

(73) Assignee: Novocure GmbH, Root D4 (CH)

(21) Appl. No.: 18/617,886

(22) Filed: Mar. 27, 2024

Related U.S. Application Data
(60) Provisional application No. 63/456,104, filed on Mar. 31, 2023.

(51) Int. Cl. A61N 1/40

(52) U.S. Cl. CPC A61N 1/40

(57) ABSTRACT

When transducer arrays (i.e., arrays of electrode elements) are used to apply alternating electric fields (e.g., tumor treating fields or TTFields) to a subject's body, the subject may experience electrosensation. This electrosensation can be ameliorated by changing the way the voltage increases from zero to its peak when the AC voltage is initially applied to the transducer arrays.

### CROSS REFERENCE TO RELATED APPLICATIONS

This Application claims the benefit of U.S. Provisional Application 63/456,104, filed Mar. 31, 2023, which is incorporated herein by reference in its entirety.

### BACKGROUND

Tumor Treating Fields (TTFields) therapy is a proven approach for treating tumors using alternating electric fields at frequencies between 50 kHz and 1 MHz (e.g., 150-200 kHz). Because the effectiveness of TTFields depends on the direction of the alternating field with respect to the longitudinal axis of the dividing tumor cells, it is preferable to apply the alternating electric fields in different orientations at different times during the course of treatment.

In the prior art Optune® system, TTFields are delivered to patients via four transducer arrays that are placed on the patient's skin near the tumor. The transducer arrays are arranged in two pairs, with one pair of transducer arrays positioned to the left and right of the tumor, and the other pair of transducer arrays positioned anterior and posterior to the tumor. Each transducer array is connected via a multi-wire cable to an AC signal generator. The AC signal generator (a) sends an AC current through the anterior/posterior (A/P) pair of transducer arrays for 1 second, which induces an electric field with a first orientation through the tumor; then (b) sends an AC current through the left/right (L/R) pair of arrays for 1 second, which induces an electric field with a second orientation through the tumor. The system then repeats steps (a) and (b) for the duration of the treatment, which repeatedly switches the orientation of the electric field.

U.S. Pat. No. 7,565,206 (which is incorporated herein by reference in its entirety), describes another approach for varying the orientation of the alternating electric field. More specifically, when a first sinusoid is applied to the A/P pair of transducer arrays and a second sinusoid at the same frequency is applied to the L/R transducer arrays, and when the first and second sinusoids are 90° out of phase with each other, the orientation of the electric field will continuously rotate through 360° during the course of treatment.

Alternating electric fields can also be used to treat medical conditions other than tumors. For example, as described in U.S. Pat. No. 10,967,167 (which is incorporated herein by reference in its entirety), alternating electric fields can be used to increase the permeability of the blood brain barrier so that, e.g., chemotherapy drugs can reach the brain.

When treating a subject using alternating electric fields, higher amplitudes are strongly associated with higher efficacy of treatment. However, as the amplitude of the alternating electric field increases, and/or as the frequency of the alternating electric field decreases (e.g., to the vicinity of 100 kHz), some subjects experience an electrosensation effect. This electrosensation could be, for example, a vibratory sensation, paresthesia, and/or a twitching or contraction sensation of muscle fibers, or a flicker of light in the eyes (phosphene). Electrosensation may discourage some subjects from continuing their treatment using alternating electric fields. Furthermore, electrosensation can limit the amplitude of the alternating electric fields that can comfortably be applied to the given subject, which in turn can limit the efficacy of the treatment.

### SUMMARY OF THE INVENTION

One aspect of the invention is directed to a first method of selectively destroying or inhibiting the growth of rapidly dividing cells located within a target region of a subject's body. The first method comprises imposing an AC electric field in the target region with a field orientation that rotates with respect to the target region. The electric field has a frequency between 50 kHz and 1 MHz, and an amplitude that increases from an initial level to a final level over the course of at least 0.1 s.

In some instances of the first method, when the amplitude of the electric field is at the final level, the electric field has a field strength of at least 1 V/cm in at least a portion of the target region. In some instances of the first method, the electric field is imposed in the target region via insulated electrodes. In some instances of the first method, the electric field has an amplitude that increases from the initial level to the final level over the course of at least 0.3 s.

In some instances of the first method, the electric field has an amplitude that increases from the initial level to the final level over the course of at least 1 s. Optionally, in these instances, when the amplitude of the electric field is at the final level, the electric field has a field strength of at least 5 V/cm in at least a portion of the target region.

In some instances of the first method, the electric field has an amplitude that increases from the initial level to the final level over the course of at least 0.3 s. When the amplitude of the electric field is at the final level, the electric field has a field strength of at least 5 V/cm in at least a portion of the target region. The frequency of the electric field is between 80 kHz and 300 kHz.

In some instances of the first method, the frequency of the electric field is between 80 kHz and 300 kHz. In some instances of the first method, the electric field has an amplitude that remains at the final level for at least 100 s. In some instances of the first method, the rotation of the AC electric field is accomplished by simultaneously applying AC voltages having different phases to at least three electrodes. In some instances of the first method, the increase in amplitude from the initial level to the final level is linear.

Another aspect of the invention is directed to a first apparatus for selectively destroying or inhibiting the growth of rapidly dividing cells located within a target region of a subject's body. The first apparatus comprises at least three electrodes, each of which has a surface configured for placing against the subject's body; and an AC voltage source having at least three outputs, each output being electrically connected to a respective one of the electrodes. Each of the at least three outputs has a frequency between 50 kHz and 1 MHz, and each of the at least three outputs has an amplitude that increases from an initial level to a final level of at least 50 V RMS over the course of at least 0.1 s.

In some embodiments of the first apparatus, each of the at least three outputs has an amplitude that remains at the final level for at least 30 s. In some embodiments of the first apparatus, each of the at least three electrodes comprises a conductive substrate, and each of the surfaces configured for placing against the subject's body comprises an insulating material having a dielectric constant of at least 20 disposed on a respective conductive substrate.

In some embodiments of the first apparatus, each of the at least three outputs has an amplitude that increases from the initial level to the final level over the course of at least 0.3 s, and the final level is at least 100 V RMS.

In some embodiments of the first apparatus, each of the at least three outputs has an amplitude that increases from the initial level to the final level over the course of at least 1 s, and the final level is at least 100 V RMS. Optionally, in these embodiments, each of the at least three outputs has a frequency between 80 kHz and 300 kHz.

In some embodiments of the first apparatus, each of the at least three outputs has a frequency between 80 kHz and 300 kHz. In some embodiments of the first apparatus, each of the at least three outputs has an amplitude that remains at the final level for at least 100 s.

In some embodiments of the first apparatus, the AC voltage source has a first output at a given frequency, a second output at the given frequency that is offset by 120° with respect to the first output, and a third output at the given frequency that is offset by 240° with respect to the first output. In some embodiments of the first apparatus, the AC voltage source has a first output at a given frequency, a second output at the given frequency that is offset by 90° with respect to the first output, a third output at the given frequency that is offset by 180° with respect to the first output, and a fourth output at the given frequency that is offset by 270° with respect to the first output.

In some embodiments of the first apparatus, the AC voltage source simultaneously applies sinusoidal signals at a first frequency to each of the at least three outputs, wherein the signals applied to each of the at least three outputs are modulated by sinusoids at a second frequency that is at least ten times lower than the first frequency, and the modulating sinusoids are phase shifted. In some embodiments of the first apparatus, the increase in amplitude from the initial level to the final level is linear.

### BRIEF DESCRIPTION OF THE DRAWINGS

FIG. 1 is a block diagram of a system for applying TTFields to a target region in a subject's body.

FIG. 2 depicts an example of an amplitude trajectory that can result in noticeable electrosensation.

FIG. 3 depicts one example of an amplitude trajectory that can be used to ameliorate electrosensation.

FIG. 4 depicts another example of an amplitude trajectory that can be used to ameliorate electrosensation.

FIG. 5 depicts four examples of non-linear amplitude-increasing trajectories that can be used to ameliorate electrosensation.

FIG. 6 is a block diagram of another system for applying TTFields to a target region in a subject's body.

Various embodiments are described in detail below with reference to the accompanying drawings, wherein like reference numerals represent like elements.

### DESCRIPTION OF THE PREFERRED EMBODIMENTS

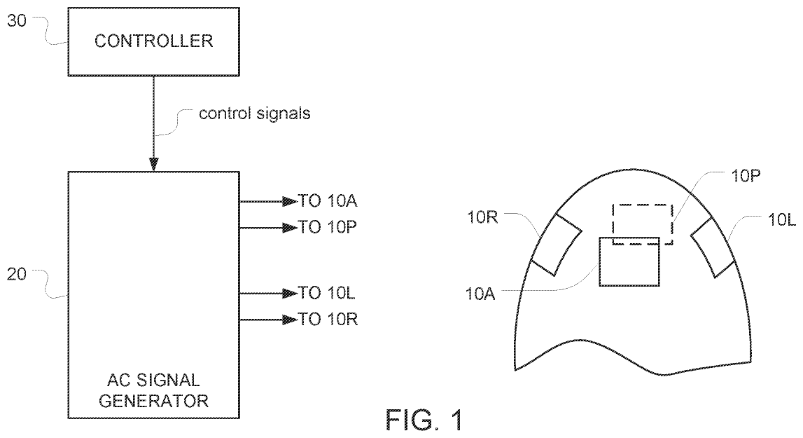

FIG. 1 is a block diagram of a system for applying TTFields to a target region in a subject's body. The system includes an AC signal generator 20 that is designed to generate first and second AC outputs at a frequency between 50 kHz and 10 MHz (e.g., 50 kHz-1 MHz, 50-500 kHz, 80-300 kHz, or 150-250 kHz). The AC signal generator 20 applies a first sinusoid to the A/P pair of electrode elements 10A/10P, and also applies a second sinusoid at the same frequency to the L/R electrode elements 10L/10R. The surfaces of each of these electrode elements are configured for placing against the subject's body. Applying AC voltages to the electrode elements 10A/10P/10L/10R will impose an alternating electric field in the target region. When the electrode elements 10A/10L/10P/10R are positioned at 90° intervals around a target body part, and the first and second sinusoids are 90° out of phase with each other, the orientation of the electric field in the target region will rotate through 360° continuously and repeatedly during the course of treatment.

Because the first sinusoid is being applied between the 10A output and the 10P output, the signal at 10P will be 180° out of phase with respect to the signal at 10A. Similarly, because the second sinusoid is being applied between the 10L output and the 10R output, the signal at 10L will be 180° out of phase with respect to the signal at 10R. And because the second sinusoid is 90° out of phase with respect to the first sinusoid, the signal at 10L will be 90° out of phase with respect to the signal at 10A, and the signal at 10R will be 270° out of phase with respect to the signal at 10A.

In some embodiments, each of the electrode elements comprises a conductive substrate, and each of the surfaces configured for placing against the subject's body comprises an insulating material with a dielectric constant of at least 20 disposed on a respective conductive substrate.

Electrosensation is believed to originate from interactions between the alternating electric fields and nerve cells or fibers (i.e., neurons or axons) that are positioned near or adjacent to the electrode elements. The inventors have determined that electrosensation is not a problem during steady-state application of an AC voltage to a given pair of electrode elements, or when the AC voltage turns off or ramps down. Instead, electrosensation appears to only be a problem when the AC voltage turns on or ramps up. The inventors have also determined that electrosensation is strongly dependent on the trajectory of how the AC voltage increases from zero to its peak when the AC voltage is switched on.

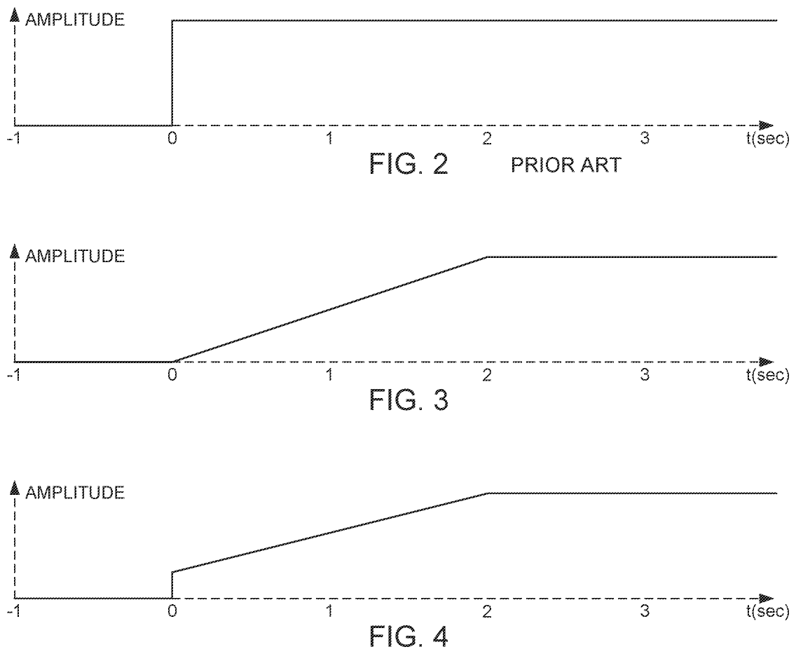

More specifically, if the amplitude of the AC voltages that are applied to the subject's body jumps immediately from zero to its peak level when the system is turned on at t=0, as depicted in FIG. 2, the electrosensation can be very noticeable. But if the amplitude of the AC voltages that are applied to the subject body rises up more slowly to its peak level, electrosensation will not occur (or will at least be dramatically reduced).

The embodiments described herein take advantage of this phenomena to eliminate or reduce electrosensation by avoiding the fast-rising situation depicted in FIG. 2, and configuring the system so that the amplitude of the AC voltages that are applied to the subject's body do not rise too quickly. More specifically, the trajectory of how the AC voltage increases from zero to its peak in the FIG. 1 embodiment differs from the instant-on trajectory depicted in FIG. 2 in a manner that prevents electrosensation from occurring, or at least reduces the level of electrosensation.

The AC signal generator 20 is configured to generate two out-of-phase sinusoidal outputs with amplitudes that depend on a state of at least one control input. A controller 30 sends sequential control signals to the at least one control input, and these control signals cause the AC signal generator 20 to adjust its output amplitude accordingly. The combination of the controller 30 and the AC generator 20 can therefore be used to generate any of the amplitude trajectories described herein. Note that although FIG. 1 depicts the controller 30 and the AC signal generator 20 as two distinct blocks, those two blocks may be integrated into a single hardware device.

FIG. 3 depicts one example of an amplitude trajectory that can be used to ameliorate electrosensation. In this example, the controller 30 sends a first series of control signals to the AC generator 20. This first series of control signals instructs the AC generator 20 to ramp up its output amplitude linearly over the course of two seconds until the final voltage is reached. In some embodiments, the final voltage is at least 50 V RMS (e.g., at least 100 V RMS or 100-200 V RMS), and this induces an electric field with a field strength of at least 1 V/cm (e.g., at least 5 V/cm, 1-10 V/cm, or 5-10 V/cm) in at least a portion of the target region. And because the AC voltages that are applied to the subject's body rise slowly to their final level in this embodiment, electrosensation will not occur (or will at least be dramatically reduced). After the ramp-up period, the output of the AC signal generator 20 remains constant for an extended period of time (e.g., for at least 30 s, at least 60 s, at least 100 s, etc.).

FIG. 4 depicts another example of an amplitude trajectory that can be used to ameliorate electrosensation. In this example, the controller 30 sends a second series of control signals to the AC generator 20. This second series of control signals instructs the AC generator 20 to set its output amplitude to a relatively low initial level at t=0, then ramp up its output amplitude linearly over the course of two seconds until the final voltage is reached. The initial level is selected to be below the threshold that causes electrosensation (e.g., below 20 V RMS). In some embodiments, the final voltage is at least 50 V RMS (e.g., at least 100 V RMS or 100-200 V RMS), and this induces an electric field with a field strength of at least 1 V/cm (e.g., at least 5 V/cm, 1-10 V/cm, or 5-10 V/cm) in at least a portion of the target region. And here again, because the AC voltages that are applied to the subject's body rise slowly to their final level, electrosensation will not occur (or will at least be dramatically reduced). After the ramp-up period, the output of the AC signal generator 20 remains constant for an extended period of time as described above in connection with FIG. 3.

Note that while the examples depicted in FIGS. 3 and 4 both have a ramp-up period of 2 seconds, the ramp-up period could be different (e.g., at least 0.1 s, at least 0.3 s, at least 1 s, at least 3 s, at least 10 s, etc.). Moreover, the linear ramps depicted in FIGS. 3 and 4 are not the only amplitude trajectories that can be used to prevent or ameliorate electrosensation. To the contrary, any of a wide variety of amplitude trajectories can be used, as long as the AC voltages that are applied to the subject's body increase slowly enough to avoid or at least minimize electrosensation.

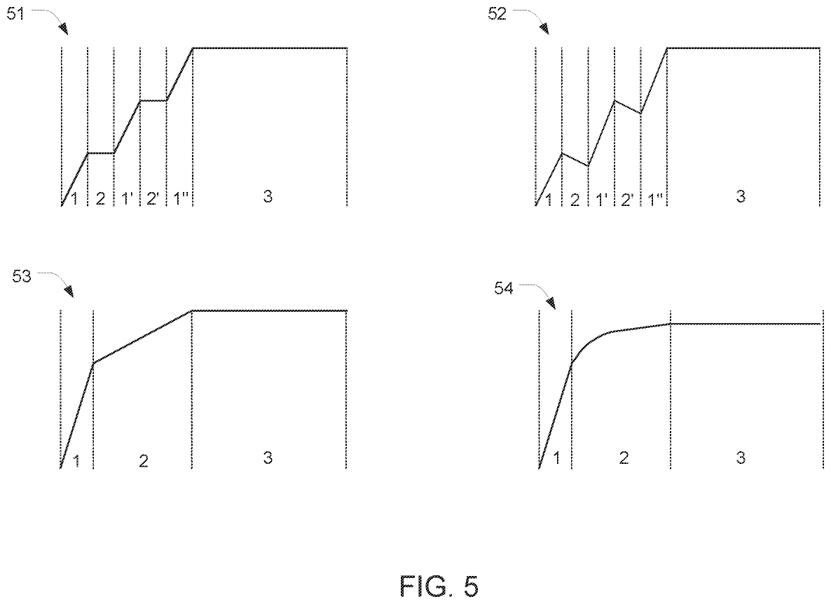

FIG. 5 depicts four examples of non-linear amplitude-increasing trajectories that can be used to ameliorate electrosensation. In the first example 51, the amplitude increases during interval 1, then remains constant during interval 2, then increases further during interval 1′, then remains constant during interval 2′, then increases further during interval 1″. To generate this amplitude trajectory, the controller 30 sequentially sends control signals (e.g., once every 1, 2, 5, 10, 20, 50, or 100 ms) to the AC signal generator 20. When the AC signal generator 20 receives these control signals, it will generate an output with an amplitude trajectory that resembles trace 51. Each of the intervals 1, 1′, 1″, 2, 2′ can be, for example, between 10 ms and 10 s long. The rationale for including the intervals 2, 2′ in the trajectory is to allow the subject to get used to a given voltage setting before the voltage is increased further. In some embodiments, each of these intervals is at least 20 ms, at least 50 ms, or at least 100 ms long. After the amplitude-increasing trajectory has occurred, the output of the AC signal generator 20 remains constant at its final value (e.g., at least 50 V RMS, at least 100 V RMS, etc.) for an extended period of time as described above in connection with FIG. 3.

A second example 52 is similar to the first example 51, except that instead of remaining constant during the intervals 2 and 2′, the amplitude of the AC signal generator 20 decreases during intervals 2 and 2′. To generate this amplitude trajectory, the controller 30 sequentially sends control signals (e.g., once every 1, 2, 5, 10, 20, 50, or 100 ms) to the AC signal generator 20. When the AC signal generator 20 receives these control signals, it will generate an output with an amplitude trajectory that resembles trace 52. Each of the intervals 1, 1′, 1″, 2, 2′ can be, for example, between 10 ms and 10 s long. After the amplitude-increasing trajectory has occurred, the output of the AC signal generator 20 remains constant at its final value for an extended period of time as described above in connection with trace 51.

In a third example 53, the controller 30 sequentially sends control signals to the AC signal generator 20 so that the amplitude of the AC signal generator 20 will increase linearly at a first rate during interval 1 (which can be, for example, between 100 ms and 10 s long), and increase linearly at a second rate during interval 2 (which can also be, for example, between 100 ms and 10 s long). After the amplitude-increasing trajectory has occurred, the output of the AC signal generator 20 remains constant at its final value for an extended period of time as described above in connection with trace 51.

In a fourth example 54, the controller 30 sequentially sends control signals to the AC signal generator 20 so that the amplitude of the AC signal generator 20 will increase at a first linear rate during interval 1 (which can be, for example, between 100 ms and 10 s long), and increase at a second non-linear rate during interval 2 (which can also be, for example, between 100 ms and 10 s long). Here again, after the amplitude-increasing trajectory has occurred, the output of the AC signal generator 20 remains constant at its final value for an extended period of time as described above in connection with trace 51.

The four examples 51-54 provided above are not exhaustive. To the contrary, a wide variety of alternative amplitude-increasing trajectories that prevent the amplitude from increasing too rapidly in order to ameliorate electrosensation can be readily envisioned.

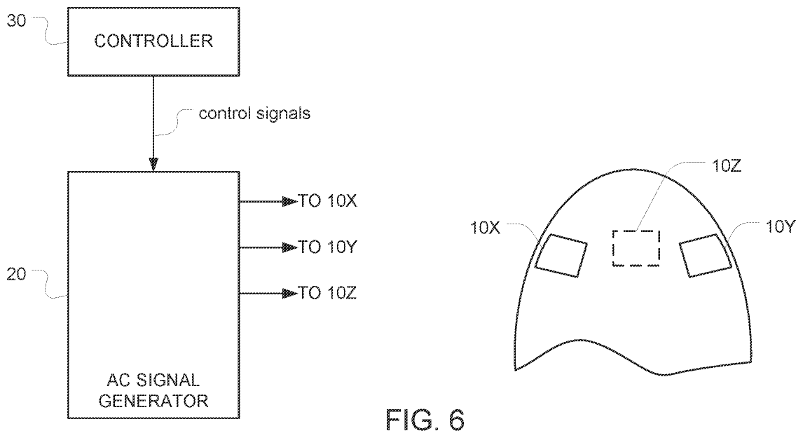

FIG. 6 is a block diagram of another system for applying TTFields to a target region in a subject's body. This system is similar to the FIG. 1 system described above, except that the FIG. 6 embodiment uses a three-phase approach to implement field rotation instead of the sine-cos approach described above in connection with FIG. 1. The FIG. 6 embodiment uses an AC signal generator 20 that is designed to generate three AC outputs at a frequency between 50 kHz and 10 MHz (e.g., 50 kHz-1 MHz, 50-500 kHz, 80-300 kHz, or 150-250 kHz). The three outputs are 120° out of phase with each other, and are applied to three sets of electrode elements 10X/10Y/10Z that are configured for placing against the subject's body. When the electrode elements 10X/10Y/10Z are positioned at 120° intervals around a target body part, applying the three-phase signals to those electrode elements will impose an alternating electric field in the target region with an orientation that rotates through 360° continuously and repeatedly during the course of treatment.

Except for the distinction that this FIG. 6 embodiment uses three-phase field rotation instead of the sine/cosine based field rotation described above in connection with FIG. 1, the operation of this FIG. 6 embodiment is similar to the operation of the FIG. 1 embodiment described above.

Finally, in some anatomic locations, the electrode elements are not positioned on the subject's skin. Instead, the electrode elements are implanted into the subject's body (e.g., just beneath the subject's skin) so that application of an AC voltage between the electrode elements will impose the alternating electric fields in a target region of the subject's body.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of selectively destroying or inhibiting the growth of rapidly dividing cells located within a target region of a subject's body, comprising the steps of:
imposing an AC electric field in the target region with a field orientation that rotates with respect to the target region, wherein the electric field has a frequency between 50 kHz and 1 MHz,
wherein the electric field has an amplitude that increases from an initial level to a final level over the course of at least 0.1 s.

2. The method of claim 1, wherein when the amplitude of the electric field is at the final level, the electric field has a field strength of at least 1 V/cm in at least a portion of the target region.

3. The method of claim 1, wherein the electric field is imposed in the target region via insulated electrodes.

4. The method of claim 1, wherein the electric field has an amplitude that increases from the initial level to the final level over the course of at least 1 s.

5. The method of claim 4, wherein when the amplitude of the electric field is at the final level, the electric field has a field strength of at least 5 V/cm in at least a portion of the target region.

6. The method of claim 1, wherein the electric field has an amplitude that increases from the initial level to the final level over the course of at least 0.3 s, wherein when the amplitude of the electric field is at the final level, the electric field has a field strength of at least 5 V/cm in at least a portion of the target region, and wherein the frequency of the electric field is between 80 kHz and 300 kHz.

7. The method of claim 1, wherein the frequency of the electric field is between 80 kHz and 300 kHz.

8. The method of claim 1, wherein the electric field has an amplitude that remains at the final level for at least 100 s.

9. The method of claim 1, wherein the rotation of the AC electric field is accomplished by simultaneously applying AC voltages having different phases to at least three electrodes.

10. The method of claim 1, wherein the increase in amplitude from the initial level to the final level is linear.

11. An apparatus for selectively destroying or inhibiting the growth of rapidly dividing cells located within a target region of a subject's body, the apparatus comprising:
at least three electrodes, each of which has a surface configured for placing against the subject's body; and
an AC voltage source having at least three outputs, each output being electrically connected to a respective one of the electrodes,
wherein each of the at least three outputs has a frequency between 50 kHz and 1 MHz, and
wherein each of the at least three outputs has an amplitude that increases from an initial level to a final level of at least 50 V RMS over the course of at least 0.1 s.

12. The apparatus of claim 11, wherein each of the at least three outputs has an amplitude that remains at the final level for at least 30 s.

13. The apparatus of claim 11, wherein each of the at least three electrodes comprises a conductive substrate, and
wherein each of the surfaces configured for placing against the subject's body comprises an insulating material having a dielectric constant of at least 20 disposed on a respective conductive substrate.

14. The apparatus of claim 11, wherein each of the at least three outputs has an amplitude that increases from the initial level to the final level over the course of at least 1 s, and
wherein the final level is at least 100 V RMS.

15. The apparatus of claim 14, wherein each of the at least three outputs has a frequency between 80 kHz and 300 kHz.

16. The apparatus of claim 11, wherein each of the at least three outputs has a frequency between 80 kHz and 300 kHz.

17. The apparatus of claim 11, wherein the AC voltage source has a first output at a given frequency, a second output at the given frequency that is offset by 120° with respect to the first output, and a third output at the given frequency that is offset by 240° with respect to the first output.

18. The apparatus of claim 11, wherein the AC voltage source has a first output at a given frequency, a second output at the given frequency that is offset by 90° with respect to the first output, a third output at the given frequency that is offset by 180° with respect to the first output, and a fourth output at the given frequency that is offset by 270° with respect to the first output.

19. The apparatus of claim 11, wherein the AC voltage source simultaneously applies sinusoidal signals at a first frequency to each of the at least three outputs, wherein the signals applied to each of the at least three outputs are modulated by sinusoids at a second frequency that is at least ten times lower than the first frequency, and the modulating sinusoids are phase shifted.

20. The apparatus of claim 11, wherein the increase in amplitude from the initial level to the final level is linear.
